(19) United States
(12) Patent Application Publication
(10) Pub. No.: US 2017/0189107 A1
(43) Pub. Date: Jul. 6, 2017

(54) MODULAR DISC ARRAY FOR MINIMALLY INVASIVE MEDICAL DEVICE

(71) Applicants: Boston Scientific Scimed Inc., Maple Grove, MN (US); Regents of the University of Minnesota, Minneapolis, MN (US)

(72) Inventors: David R. Wulfman, Minneapolis, MN (US); Daniel J. Foster, Lino Lakes, MN (US); Matthew Hein, Eden Prairie, MN (US); Kevin R. Poppe, New Brighton, MN (US); Bethanie H. Stadler, Shoreview, MN (US)

(21) Appl. No.: 15/396,677

(22) Filed: Jan. 1, 2017

Related U.S. Application Data
(60) Provisional application No. 62/274,352, filed on Jan. 3, 2016.

(51) Int. Cl. A61B 18/14

(52) U.S. Cl. CPC A61B 18/1492; A61B 2017/00314

(57) ABSTRACT

In certain embodiments, an apparatus includes a stacked array of discs electrically coupled together where each disc includes an electrical component. In certain embodiments, a minimally-invasive medical device includes a stack of transducer platforms positioned within the minimally-invasive medical device. Each platform includes a plurality of electrical components.

### CROSS-REFERENCE TO RELATED APPLICATION

This application claims priority to Provisional Application No. 62/274,352, filed Jan. 3, 2016, which is herein incorporated by reference in its entirety.

### TECHNICAL FIELD

The present invention relates to medical devices and methods for positioning electrical components within medical devices.

### BACKGROUND

Minimally-invasive medical devices like catheters are devices inserted into patients to assist with or perform medical procedures and diagnostics.

### SUMMARY

In Example 1, an apparatus includes a stacked array of discs electrically coupled together where each disc includes an electrical component.

In Example 2, the apparatus of Example 1, wherein the electrical component is a sensor.

In Example 3, the apparatus of any of Examples 1-2, wherein each of the discs includes multiple sensors.

In Example 4, the apparatus of any of Examples 2-3, wherein the sensors include magnetoresistive elements.

In Example 5, the apparatus of any of Examples 2-4, wherein each sensor is configured to sense magnetic fields in a direction different than the other sensors.

In Example 6, the apparatus of any of Examples 1-5, wherein one of the discs includes at least two different types of sensors.

In Example 7, the apparatus of any of Examples 1-6, wherein one disc includes sensors having magnetoresistive elements and another disc includes temperature sensors.

In Example 8, the apparatus of any of Examples 2-7, wherein the sensors are mounted to a substrate of each disc.

In Example 9, the apparatus of any of Examples 2-7, wherein the sensors are embedded in each disc.

In Example 10, the apparatus of any of Examples 1-9, wherein the disc includes optical components.

In Example 11, the apparatus of any of Examples 1-10, wherein each disc includes a plurality of electrical components.

In Example 12, the apparatus of any of Examples 1-11, wherein the stacked disc array includes four discs.

In Example 13, a minimally-invasive medical device includes a body forming an aperture. The device also includes a plurality of transducer platforms positioned within the aperture and electrically coupled together. Each platform includes an electrical component. At least one of the platforms facilitates electrical communication between the other plurality of platforms and off-platform electrical components associated with the minimally-invasive medical device.

In Example 14, the device of Example 13, wherein the electrical component is a transducer.

In Example 15, the device of any of Examples 13-14, wherein the platforms are one of square-shaped, disc-shaped, and hexagon-shaped.

In Example 16, a minimally-invasive medical device includes a stack of discs positioned at a distal end of the minimally-invasive medical device. Each disc includes a plurality of electrical components.

In Example 17, the device of Example 16, wherein the discs are electrically or optically coupled to each other.

In Example 18, the device of any of Examples 16-17, wherein at least some of the plurality of electrical components are transducers.

In Example 19, the device of Example 18, wherein at least some of the transducers include magnetoresistive elements.

In Example 20, the device of any of Examples 18-19, wherein at least some of the transducers are temperature sensors.

In Example 21, the device of any of Examples 18-19, wherein each transducer is configured to sense magnetic fields in a direction different than the other transducers.

In Example 22, the device of any of Examples 16-21, further including a cap and body forming an aperture housing the stack of discs.

In Example 23, the device of Example 22, further including a retainer that physically couples the discs, cap, and body together.

In Example 24, the device of Example 23, wherein the retainer extends through center apertures formed in each of the discs.

In Example 25, the device of any of Examples 16-24, wherein each disc includes a plurality of protrusions that are sized to fit into holes defined by an adjacent disc.

In Example 26, the device of any of Examples 16-25, wherein the plurality of electrical components are mounted to a disc's substrate.

In Example 27, the device of any of Examples 16-26, wherein the plurality of electrical components are embedded in each disc.

In Example 28, the device of any of Examples 16-27, wherein the discs comprise silicon.

In Example 29, the device of any of Examples 16-28, wherein the plurality of electrical components includes at least one of the following: a battery, capacitor, amplifier, analog-to-digital converter, signal processor, wireless communicator, radiofrequency ablator, or cryo-ablators.

In Example 30, the device of any of Examples 16-29, wherein the plurality of electrical components are electrically coupled to each other.

In Example 31, the device of any of Examples 16-29, wherein each electrical component is independently electrically coupled to a most proximal disc of the stack.

In Example 32, the device of any of Examples 16-31, wherein a most proximal disc of the stack facilities electrical communication between the stack of discs and off-disc electrical components associated with the medical device.

In Example 33, a catheter includes a plurality of sensing structures coupled together and positioned within the catheter. Each sensing structure includes a plurality of sensors having a transducer.

In Example 34, the catheter of Example 33, wherein the plurality of sensing structure are positioned within a hollow cavity formed by the catheter and at a distal end of the catheter.

In Example 35, the catheter of any of Examples 33-34, wherein the sensing structures are disc-shaped and stacked together.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a partial exploded view of a distal portion of a medical device, in accordance with certain embodiments of the present disclosure.

FIG. 2 shows a partial exploded view of a top portion of a medical device, in accordance with certain embodiments of the present disclosure.

FIG. 3a shows a partial view of an assembled medical device, in accordance with certain embodiments of the present disclosure.

FIG. 3b shows a partial section view of the assembled medical device of FIG. 3a and an array of discs positioned within the device, in accordance with certain embodiments of the present disclosure.

FIG. 3c shows a top view of one of the discs in the array of FIG. 3b, in accordance with certain embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Minimally-invasive medical devices can be provisioned with various electrical components such as transducers to enhance functionality. Because medical devices are used for a wide variety of procedures, a device's desired range of functionality may vary from device to device. For example, catheters may benefit when provisioned with magnetic field sensors, which can assist with tracking and navigating catheters during medical procedures. Irrigated catheters—which use a fluid to control the catheter's temperature—may further benefit when additionally provisioned with temperature sensors. However, because the size of devices like catheters is limited, fitting additional components like transducers into devices can be challenging. Features of the present disclosure are accordingly directed to methods and devices for positioning electrical components within medical devices.

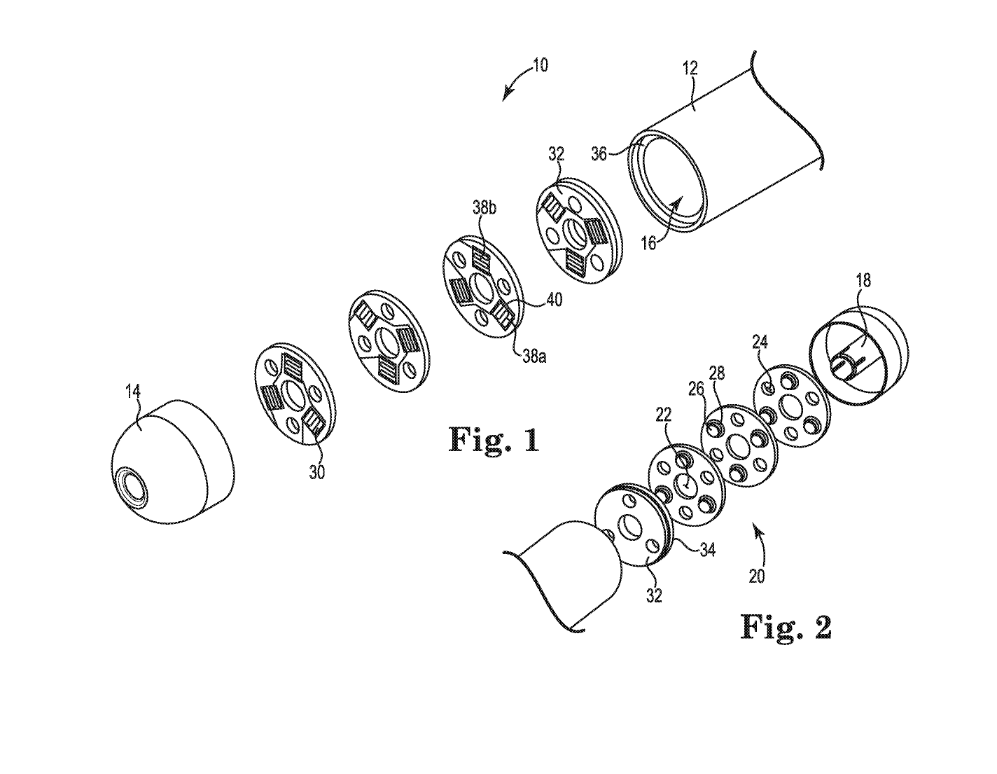

FIGS. 1 and 2 show partial exploded views of a tip 10 of a medical device such as a catheter. Although a catheter is used as an example, the present disclosure is applicable to a wide variety of medical devices. The catheter includes a body 12 and cap 14 that form a center aperture 16. The cap 14 is coupled to the body 12 by a retaining structure 18 shown in FIG. 2. An array of discs 20 is positioned and maintained within the center aperture 16. Each disc is shown as having a central opening 22, multiple holes 24 around the central opening 22, and multiple protrusions 26. The central opening 22 permits the retainer 18 to extend through the stacked array of discs 20 to secure the cap 14 and discs 20 to the body 12. The protrusions 26 help secure a position of one disc in relation to a disc positioned immediately adjacent. As shown in FIGS. 1 and 2, the protrusions 26 of one disc are aligned with holes 24 of an adjacent disc such that, when the discs are stacked together, the protrusions 26 fit into the holes 24. Each protrusion 26 is shown extending from another protrusion 28, which adjacent discs rest upon when stacked to create a space between the discs for positioning electrical components 30. As will be described below in more detail, the discs' electrical components 30 can be used to perform a variety of functions by using components such as transducers and sensors.

The array architecture permits modularity, which enables a wide variety of configurations of discs or platforms and respective electrical components and connections. Although discs are shown and described, components like transducers can be positioned on differently-shaped platforms. For example, an individual platform within an array of platforms could be shaped as a square, hexagon, etc. The configuration shown in FIGS. 1 and 2 includes four discs that are stacked upon one another and positioned and retained within a medical device once assembled. Each disc is shown having a central opening 22 that aligns with a central opening of the other discs in the array. Each disc also has three holes 24 that are offset from holes of an immediately-adjacent disc. The three discs positioned nearest the cap 14 have protrusions 26 on the bottom side of each disc. When assembled, the protrusions 26 of each disc fit into the holes 24 of the disc positioned immediately below. Bottom disc 32 is shown as having a rim 34 that rests upon a lip 36 in the body 12 though the bottom disc 32 could also have protrusions or other features that fit into holes or other features on the body 12.

Each disc is shown having electrical components 30 positioned on a top side of the disc. One type of electrical component could include a sensor and, more specifically, a magnetoresistive sensor. Magnetoresistive sensors can include giant- or tunneling-magnetoresistive elements, which sense magnetic fields. As mentioned above, magnetic field sensors like magnetoresistive sensors can assist with tracking and navigating medical devices during medical procedures. The discs in FIGS. 1 and 2 are shown having three groupings 38 of sensors. Each group 38 could be configured to sense magnetic fields in a particular direction. For example, the first group 38a of sensors could sense magnetic fields in a direction perpendicular to a top surface of the disc while the second group 38b could sense magnetics fields in a parallel direction and so on.

The sensors 38 are shown as being electrically connected in series with traces 40 extending to the first and third groups from a side of the disc. The traces of one disc can be electrically connected to the traces of the other discs. The bottom disc 32 can function as an electrical and communications bus that facilitates signal communication and powering of the other discs. For example, the bottom disc 32 can receive signals from the other discs and transfer those signals to off-disc electronics of the catheter for further processing and mapping of the sensed magnetic fields. The bottom disc 32 may include electrical connectors on a bottom side of the disc for electrically coupling the disc array 20 with other electronics of the catheter.

As mentioned above, the array of structures permit modularity enabling a wide variety of configurations of discs and platforms and respective electrical components and connections. FIGS. 1 and 2 show an example of just one of many uses and configurations of the disc array. For example, fewer or more than four discs can be used in an array; and fewer or more sensors could be used on an individual disc. More discs and sensors could be added if additional sensing capabilities were desired. For example, if space in a radial direction was limited, a larger number of smaller-diameter discs could be used. Discs could have a different number of holes and protrusions or even different features, like pins or adhesives, that secure one disc to another. Instead of a central opening, discs could utilize other openings or notches that enable retainers or other features to extend through the discs. For example, medical devices like catheters may be irrigated such that lines of fluid may be directed to a tip of the device; and medical devices implementing ablation may require additional electrical signals to be sent to a tip of the device. The discs can be configured to permit such features to extend through the discs to reach desired areas of the device.

A wide variety of types and combinations of electrical components can be utilized on the modular arrays. Moreover, the modular arrays can utilize optical components like waveguides and magneto-optic transducers. In addition to the previously mentioned magnetoresistive sensors, embodiments can include discs can be provisioned with sensors that measure temperature, force, acceleration, ultrasound, flow, pressure, position, radiation levels, and other parameters. For example, discs could include temperature sensors like thermistors that may be useful for measuring temperature of irrigated medical devices where temperature control is desired. Discs could be provisioned with other electrical components to be used to function as batteries, capacitors, amplifiers, analog-to-digital converters, signal processors, wireless communicators, radiofrequency ablators, cryo-ablators, and others. An individual disc could include more than one type of electrical component or sensor. An individual disc could include only magnetoresistive sensors while another disc in the array could include only temperature sensors.

Electrical components can be positioned in a wide variety of ways. For example, the disc itself could be made of silicon so electrical components may be integral with or embedded in the disc. Discs could include substrates on which electrical components like integrated circuits are mounted to. For example, the substrate could be made of glass, and sensors and supporting sensor circuitry could be embedded in a layer of aluminum oxide or other suitable materials positioned on top of the substrate. Sensor coil configurations may be deposited on a substrate and embedded in a disc.

Discs and electrical components can be electrically connected in a variety of ways. For example, discs and electrical components could be connected using wires, traces, vias, ball-grid arrays, flex circuitry, spring contacts, wafer-to-wafer bonding, and other methods to communicate electrical signals to and from each other. For disc-to-disc electrical communication, the protrusions 26 could include conductive material that physically couples with conductive materials contained in an adjacent disc's hole 24 to electrically couple the two discs. Signals of the electrical components of each disc can be combined or kept independent of other signals. Another example of disc-to-disc electrical communication includes using a flex circuit that directs various electrical signals of the discs towards the bottom disc. Moreover, optical components such as magneto-optic transducers positioned on discs can be optically coupled together.

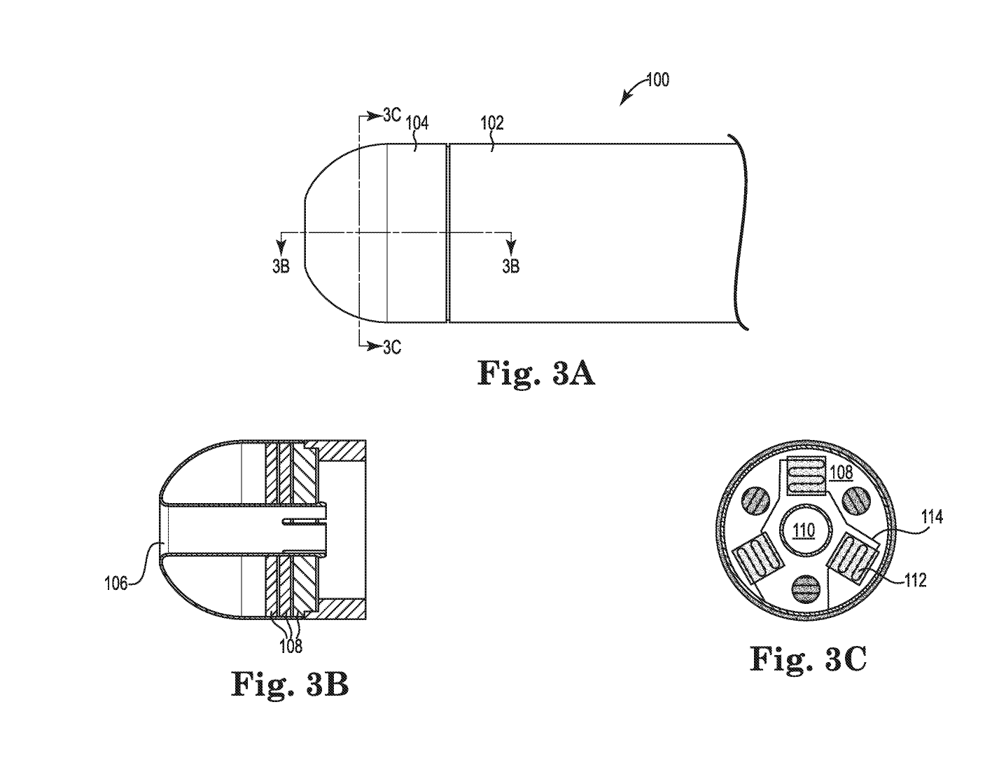

FIG. 3a shows a side view of a distal end of an assembled medical device 100 having a body 102 and cap 104. FIG. 3b shows a partial section view of the assembled medical device and an array of discs positioned within the device. FIG. 3c shows a top view of one of the discs in the array. The body 102 and cap 104 form an aperture where the discs are positioned. A retaining structure 106 integral with the cap 104 extends through three stacked discs 108 through a central opening 110 of each disc 108. Although the discs 108 are shown as being generally parallel with each other, discs may be canted such that electrical components 112 are oriented in a specific direction or angle. Additionally, electrical components 112 themselves could be oriented or positioned in a variety of positions on a disc 108. Although electrical components 112 are shown positioned on a top side of the disc 108, the disclosure is not limited to such positions. The electrical components 112 are electrically connected with traces 114 that extend from component to component and down a side of the disc 108. Signals of the electrical components could also be transmitted through the disc or other suitable electrical transmitting configurations like vias or flex circuits. Although the medical device 100 is shown as having a body 102 and cap 104 with discs positioned within the medical device, a disc could be a most distal part of the medical device. For example, a most distal disc could be arranged to form a central aperture along with the body to house other discs. Such a disc may be arranged to position an array of ultrasound transducers. Moreover, the array of discs can be positioned at various points within the medical device and not necessarily at a distal end.

Various modifications and additions can be made to the exemplary embodiments discussed without departing from the scope of the present invention. For example, while the embodiments described above refer to particular features, environments, and applications, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A minimally-invasive medical device comprising:
a stack of discs positioned at a distal end of the minimally-invasive medical device, each disc including a plurality of electrical components.

2. The minimally-invasive medical device of claim 1, wherein the discs are electrically or optically coupled to each other.

3. The minimally-invasive medical device of claim 1, wherein at least some of the plurality of electrical components are transducers.

4. The minimally-invasive medical device of claim 3, wherein at least some of the transducers include magnetoresistive elements.

5. The minimally-invasive medical device of claim 4, wherein at least some of the transducers are temperature sensors.

6. The minimally-invasive medical device of claim 4, wherein each transducer is configured to sense magnetic fields in a direction different than the other transducers. The minimally-invasive medical device of claim 1, further comprising:
a cap and body forming an aperture housing the stack of discs.

8. The minimally-invasive medical device of claim 7, further comprising:
a retainer that physically couples the discs, cap, and body together.

9. The minimally-invasive medical device of claim 8, wherein the retainer extends through center apertures formed in each of the discs.

10. The minimally-invasive medical device of claim 1, wherein each disc includes a plurality of protrusions that are sized to fit into holes defined by an adjacent disc.

11. The minimally-invasive medical device of claim 1, wherein the plurality of electrical components are mounted to a disc's substrate.

12. The minimally-invasive medical device of claim 1, wherein the plurality of electrical components are embedded in each disc.

13. The minimally-invasive medical device of claim 12, wherein the discs comprise silicon.

14. The minimally-invasive medical device of claim 1, wherein the plurality of electrical components includes at least one of the following: a battery, capacitor, amplifier, analog-to-digital converter, signal processor, wireless communicator, radiofrequency ablator, or cryo-ablators.

15. The minimally-invasive medical device of claim 1, wherein the plurality of electrical components are electrically coupled to each other.

16. The minimally-invasive medical device of claim 1, wherein each electrical component is independently electrically coupled to a most proximal disc of the stack.

17. The minimally-invasive medical device of claim 1, wherein a most proximal disc of the stack facilities electrical communication between the stack of discs and off-disc electrical components associated with the medical device.

18. A catheter comprising:
a plurality of sensing structures coupled together and positioned within the catheter, each sensing structure including a plurality of sensors having a transducer.

19. The catheter of claim 18, wherein the plurality of sensing structure are positioned within a hollow cavity formed by the catheter and at a distal end of the catheter.

20. The catheter of claim 19, wherein the sensing structures are disc-shaped and stacked together.
